(19) United States
(12) Patent Application Publication
(10) Pub. No.: US 2006/0135853 A1
(43) Pub. Date: Jun. 22, 2006

(54) Mouth speculum

(76) Inventor: Takafumi Chin, Tokyo (JP)

Correspondence Address: RICHARD M. GOLDBERG, 25 EAST SALEM STREET, SUITE 419, HACKENSACK, NJ, 07601, US

(21) Appl. No.: 11/060,127

(22) Filed: Feb. 17, 2005

(30) Foreign Application Priority Data
Dec. 21, 2004 (JP) 2004-369224

(51) Int. Cl. A61B 1/32

(52) U.S. Cl. 600/242

(57) ABSTRACT

A mouth speculum allows an intubation tube to pass through a patient's mouth in a stable condition, while offering comfort in its use. Two lip-pressing pieces curve outwardly at the left and right sides in such a manner as to become smoother toward the center in the vertical direction, and have external surfaces depressed inwardly in the left-right direction; and a handle curves upwardly or downwardly and is joined under the condition of exerting bending elasticity with respect to the two lip-pressing pieces. At least one of the two lip-pressing pieces includes a concave cut and/or a hole, to allow the intubation tube to pass through it at the front-side piece and the rear-side piece which are depressed outside in the left-right direction.

### BACKGROUND OF THE INVENTION

The present invention relates to a mouth speculum used in oral surgery or the like.

### DESCRIPTION OF THE RELATED ART

A mouth speculum (angle wider) is used in body surgery in order to keep a patient who is under general anesthesia with his/her mouth open while allowing an intubation tube for injection of an anesthetic to pass through the mouth.

Such mouth specula are classified into two types. One type (both-side type) forces a patient's mouth open by contacting with and being pressed against his/her lips elastically at the right and left sides of the lips. The other type (one-side type) forces a patient's mouth open by contacting with one side of his/her lips and being pressed against the other side with the help of a human hand.

The mouth speculum of the both-side type is superior to that of the one-side type in that no human hand is required to widen the lips.

As shown in FIGS. 5(a) and 5(b), a conventional mouth speculum of the both-side type is formed of: two lip-pressing pieces 1 which curve outwardly at the left and right sides in such a manner as to become smoother towards the center in the vertical direction, and which have external surfaces depressed inwardly in the left-right direction; and a handle 2 which curves upwardly or downwardly and which is joined under the condition of exerting bending elasticity with respect to the two lip-pressing pieces 1 (In FIG. 5(a), the handle 2 curves upwardly, and this holds true in the cases shown in FIGS. 1(a), 2(a) and 3(a) which will be described later).

More specifically, in the conventional mouth speculum of the both-side type, as shown in FIG. 5(b), the external surfaces at both sides in the left-right direction are depressed inwardly so as to cover the front side (outside) and the rear side (inside) of the lips, that is, the front piece and the rear piece are warped on the left and right sides, without any major working involved.

In this conventional structure, when an intubation tube or the like is made to pass through the mouth, it is necessary to choose between allowing the intubation tube to pass between the lip-pressing pieces 1 and teeth, and allowing the intubation tube to pass between the lips and the lip-pressing pieces 1. In the former case, it is impossible to allow the intubation tube to pass through the mouth in a stable manner, whereas in the latter case the intubation tube is pressed against the lips by the elastic force of the handle 2 of the mouth speculum, causing the patient discomfort in its use condition.

### SUMMARY OF THE INVENTION

In view of such background facts, the present invention has an object of providing a mouth speculum which allows an intubation tube to pass through a patient's mouth in a stable condition, while offering comfort in its use.

In order to achieve the object, the present invention can be:

(1) A mouth speculum comprising: two lip-pressing pieces which curve outwardly at the left and right sides in such a manner as to become smoother toward the center in the vertical direction, and which have external surfaces depressed inwardly in the left-right direction; and a handle which curves upwardly or downwardly and which is joined under the condition of exerting bending elasticity with respect to the two lip-pressing pieces, wherein at least one of the two lip-pressing pieces is provided with a cut concave to allow the intubation tube to pass through it at the front-side piece and the rear-side piece which are depressed outside in the left-right direction.

(2) A mouth speculum comprising: two lip-pressing pieces which curve outwardly at the left and right sides in such a manner as to become smoother toward the center in the vertical direction, and which have external surfaces depressed inwardly in the left-right direction; and a handle which curves upwardly or downwardly and which is joined under the condition of exerting bending elasticity with respect to the two lip-pressing pieces, wherein at least one of the two lip-pressing pieces is provided with a hole to allow the intubation tube to pass through it at the front-side piece and the rear-side piece which are depressed outside in the left-right direction.

(3) A mouth speculum comprising: two lip-pressing pieces which curve outwardly at the left and right sides in such a manner as to become smoother toward the center in the vertical direction, and which have external surfaces depressed inwardly in the left-right direction; and a handle which curves upwardly or downwardly and which is joined under the condition of exerting bending elasticity with respect to the two lip-pressing pieces, wherein at least one of the two lip-pressing pieces is provided with a combination of a cut concave and a hole to allow the intubation tube to pass through them at the front-side piece and the rear-side piece which are depressed outside in the left-right direction.

### BRIEF DESCRIPTION OF THE DRAWINGS

FIGS. 1(a) and 1(b) show the structure (1) described above; FIG. 1(a) is a front view and FIG. 1(b) is a side view.

FIGS. 2(a) and 2(b) show the structure (2) described above; FIG. 2(a) is a front view and FIG. 2(b) is a side view.

FIGS. 3(a) and 3(b) show the structure (3) described above; FIG. 3(a) is a front view and FIG. 3(b) is a side view.

FIGS. 4(a), 4(b) and 4(c) show the structures of the embodiments; FIG. 4(a) corresponds to the structure (1), FIG. 4(b) corresponds to the structure (2), and FIG. 4(c) corresponds to the structure (3).

FIGS. 5(a) and 5(b) show the structure of a mouth speculum conventionally used; FIG. 5(a) is a front view and FIG. 5(b) is a cross sectional view in the left-right direction at around the center in the vertical direction.

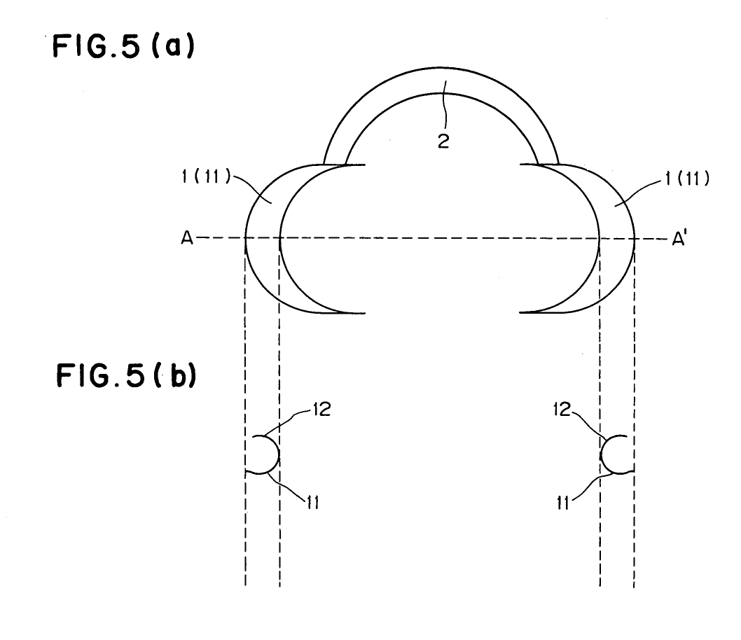

### DETAILED DESCRIPTION OF THE INVENTION

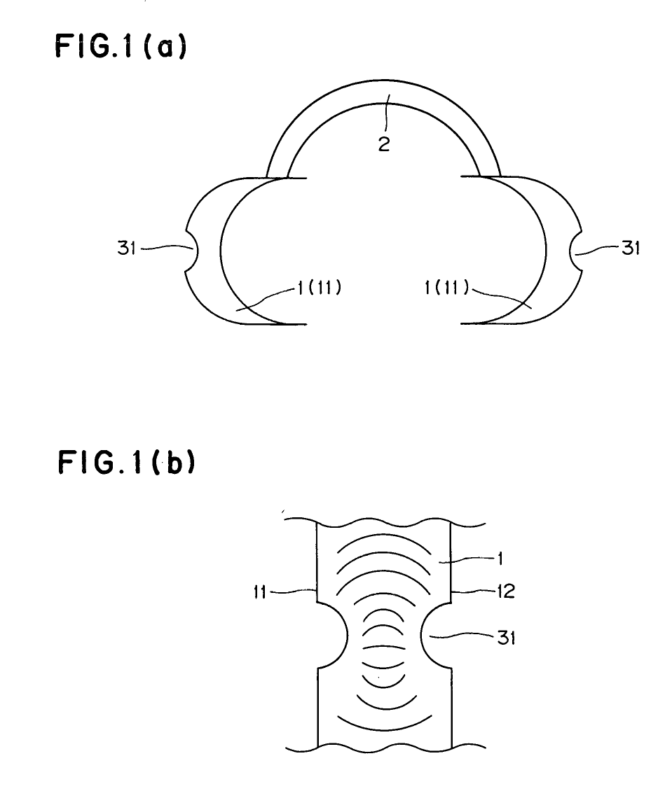

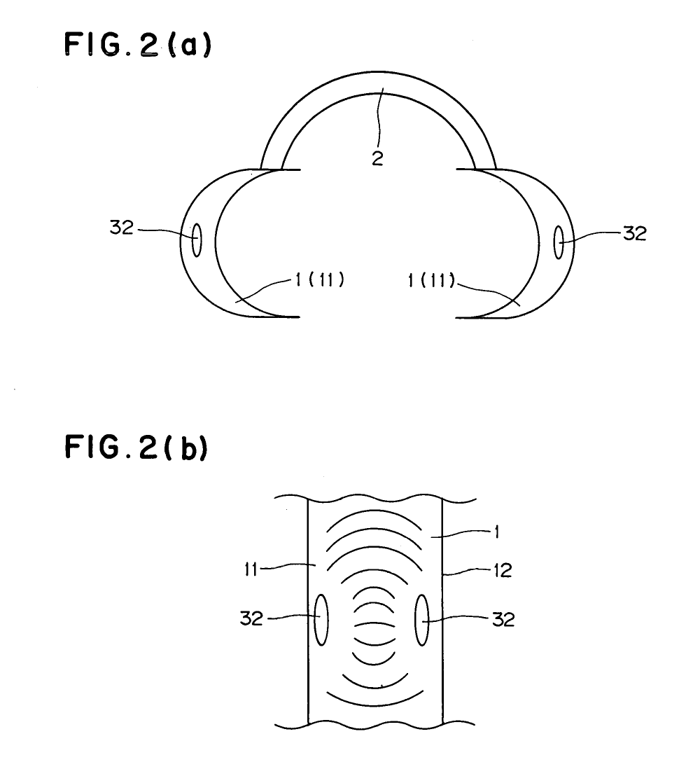

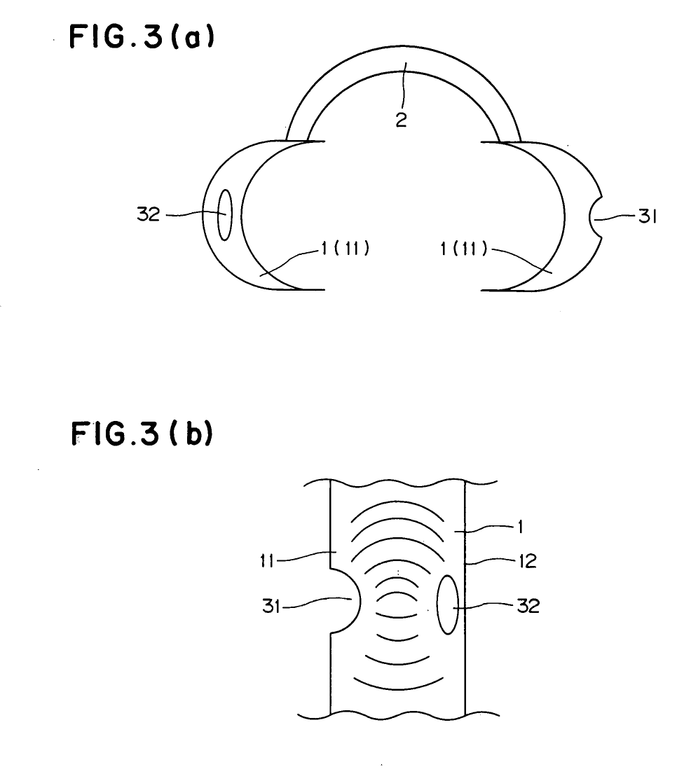

In order to allow an intubation pipe to pass through, in the structure (1), as shown in FIGS. 1(a) and 1(b), the front-side piece 11 and the rear-side piece 12 are each provided with a cut concave 31. In the structure (2), as shown in FIGS. 2(a) and 2(b), the front-side piece 11 and the rear-side piece 12 are each provided with a hole 32. In the structure (3), as shown in FIGS. 3(a) and 3(b), the front-side piece 11 and the rear-side piece 12 are each provided with a combination of the cut concave 31 and the hole 32.

The reason for providing the cut concave 31 or the hole 32 on both of the front-side piece 11 and the rear-side piece 12 is that the intubation pipe must pass through both the front-side piece 11 and the rear-side piece 12 in order to pass through the lip-pressing pieces 1.

For the solution in the structures (1), (2) and (3), at least one of the two lip-pressing pieces on both of the left and right sides is required to have either the cut concave 31 or the hole 32; however, in reality, it is preferable to provide the cut concave 31 or the hole 32 on both of the two lip-pressing pieces on both sides for the efficient passing of the intubation pipe.

In the case where the hole 32 is provided as in the structure (2), the intubation pipe must be made to pass through the hole 32 in advance. In contrast, in the case of the structure (1), the intubation pipe can be made to pass through by merely fitting the intubation pipe into the cut concave 31, without any major working involved for the passing.

In the case of providing the hole 32 in the structure (2), the intubation pipe can be easily made to pass through by setting the diameter of the hole 42 large. In addition, it is also possible to reduce the contact area between the intubation pipe and the lips by designing the holes 32 on both of the front and rear sides to be as inward as possible in the left-right direction.

The structure (3) has intermediate characteristics between the structure (1) and the structure (2) in terms of merits and demerits of these structures.

In the structure (3), it is arbitrary to select the cut concave 31 or the hole 32 to provide on the front-side piece 11 and the rear-side piece 12.

The position of the cut concave 31 or the hole 32 on the front and rear of the lip-pressing pieces 1 is not particularly restricted. However, as shown in FIGS. 1, 2 and 3, the central area or its vicinity in the vertical direction is the easiest position to allow the intubation pipe to pass through. The position to form the cut concave 31 or the hole 32 is not limited to the central area or its vicinity (note that the uppermost or lowermost areas are not large enough to form the cut concave 31 or the hole 32 because the front-side piece 11 and the rear-side piece 12 have a small width).

In the structures (1) and (3), a sense of oppression on the lips can be reduced by designing the cut concave 31 to be deeper than the diameter of the intubation pipe.

### Embodiments

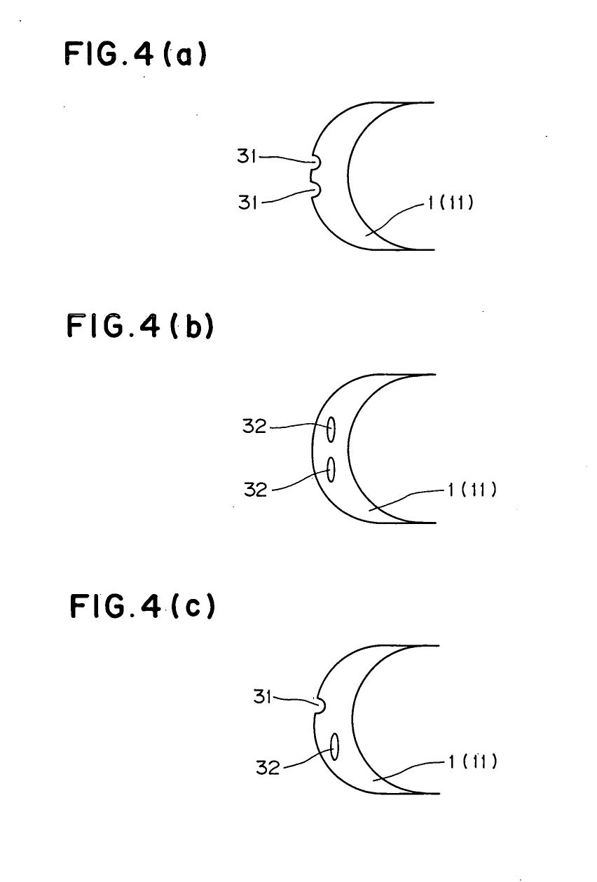

In the embodiments, as shown in FIGS. 4(a), 4(b) and 4(c), it is necessary to provide in the vertical direction a plurality of cut concaves 31, a plurality of holes 32 or a combination of the cut concave 31 and the hole 32 formed in the fore-and-aft direction (in the cases of FIGS. 4(a), 4(b) and 4(c), these are used in two sets).

Setting a plurality of such concaves or holes can allow a plurality of intubation pipes to pass though a patient's mouth, thus achieving efficient use of intubation pipes in surgery.

### EFFECTS OF THE INVENTION

According to each of the structures (1), (2) and (3), the intubation pipe can be made to pass through a patient's mouth in a stable manner by allowing it to pass through not outside the mouth speculum but through the cut concave or hole to be formed on the front-side and rear-side pieces, without an uncomfortable sense of oppression as in the case of allowing the pipe to pass through between the lips and the lip-pressing pieces.

The present invention can be used not only in the medical field but also in experiments with higher animals such as men and apes.

1. A mouth speculum comprising:
two lip-pressing pieces which curve outwardly at left and right sides thereof in such a manner as to become smoother toward a center thereof as viewed in a vertical direction thereof, and the two lip-pressing pieces have external surfaces which are depressed inwardly in a left-right direction; and
a handle which curves one of upwardly and downwardly, and which is joined under a condition of exerting bending elasticity with the two lip-pressing pieces,
at least one of the two lip-pressing pieces including a concave cut at a front-side piece and a rear-side piece thereof to allow an intubation tube to pass through the concave cut at the front-side piece and the rear-side piece, with the front-side piece and the rear-side piece forming an outwardly facing depression in the left-right direction.

2. A mouth speculum of claim 1 wherein a plurality of concave cuts are provided in a fore and aft direction in said at least one of the two lip-pressing pieces.

3. A mouth speculum comprising:
two lip-pressing pieces which curve outwardly at left and right sides thereof in such a manner as to become smoother toward a center thereof as viewed in a vertical direction thereof, and the two lip-pressing pieces have external surfaces which are depressed inwardly in a left-right direction; and
a handle which curves one of upwardly and downwardly, and which is joined under a condition of exerting bending elasticity with the two lip-pressing pieces,
at least one of the two lip-pressing pieces including a hole at a front-side piece and a rear-side piece thereof to allow an intubation tube to pass through the holes at the front-side piece and the rear-side piece, with the front-side piece and the rear-side piece forming an outwardly facing depression in the left-right direction.

4. A mouth speculum of claim 3 wherein a plurality of holes are provided in a fore and aft direction in said at least one of two lip-pressing pieces.

5. A mouth speculum comprising:
two lip-pressing pieces which curve outwardly at left and right sides thereof in such a manner as to become smoother toward a center thereof as viewed in a vertical direction thereof, and the two lip-pressing pieces have external surfaces which are depressed inwardly in a left-right direction; and
a handle which curves one of upwardly and downwardly, and which is joined under a condition of exerting bending elasticity with the two lip-pressing pieces,
at least one of the two lip-pressing pieces including a combination of a concave cut and a hole at a front-side piece and a rear-side piece thereof to allow an intubation tube to pass through the concave cut and the hole at the front-side piece and the rear-side piece, with the front-side piece and the rear-side piece forming an outwardly facing depression in the left-right in the left-right direction.

6. A mouth speculum of claim 5 wherein a plurality of concave cuts and holes are provided in a fore and aft direction in said at least one of the two lip-pressing pieces.
